(19) United States
(12) Patent Application Publication
(10) Pub. No.: US 2009/0197929 A1
(43) Pub. Date: Aug. 6, 2009

(54) Azolylmethyloxiranes, Their Use for Controlling Phytopathogenic Fungi and Agents Containing Said Compounds

(75) Inventors: Jochen Dietz, Mannheim (DE); Thomas Grote, Wachenheim (DE); Bernd Müller, Frankenthal (DE); Jan Klaas Lohmann, Ludwigshafen (DE); Jens Renner, Bad Dürkheim (DE); Sarah Ulmschneider, Bad Dürkheim (DE); Alice Glättli, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/306,339

(22) PCT Filed: Jun. 25, 2007
(86) PCT No.: PCT/EP2007/056306

§ 371 Date: Dec. 23, 2008

(30) Foreign Application Priority Data
Jul. 5, 2006 (EP) 06116611.2

(51) Int. Cl. A01N 43/64; C07D 249/08; A01P 3/00

(52) U.S. Cl. 514/383; 548/262.2

(57) ABSTRACT

The present invention relates to azolylmethyloxiranes of the general formula I

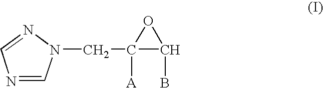

in which
A or B is a 5-membered heteroaryl selected from the group consisting of thienyl, furyl, pyrrolyl, pyrazolyl, imidazolyl, oxazolyl, thiazolyl, isoxazolyl and isothiazolyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
and the respective other substituent
A or B is phenyl which is optionally substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
and to the plant-compatible acid addition salts or metal salts thereof, to the use of the compounds of the formula I for controlling phytopathogenic fungi and to compositions comprising these compounds.

The present invention relates to azolylmethyloxiranes of the general formula I

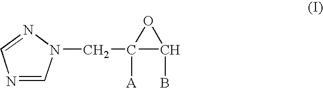

in which
A or B is a 5-membered heteroaryl selected from the group consisting of thienyl, furyl, pyrrolyl, pyrazolyl, imidazolyl, oxazolyl, thiazolyl, isoxazolyl and isothiazolyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
   and the respective other substituent
A or B is phenyl which is optionally substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
   and to the plant-compatible acid addition salts or metal salts thereof.

Furthermore, the invention relates to the use of the compounds of the formula I for controlling phytopathogenic fungi and to compositions comprising these compounds.

Azolylmethyloxiranes, their preparation and their use in crop protection are known, for example, from EP-A 0 094 564 and EP-A 0 196 038.

Azolylmethyloxiranes which carry a hetaryl substituent on the oxirane ring are known from EP-A 0 421 125.

The azolylmethyloxiranes described already have good to very good fungicidal activity against a number of pathogens; however, it was the object of the present invention to provide novel azolylmethyloxiranes having improved fungicidal activity.

This object was achieved with the compounds of the formula I described at the outset. Owing to the basic character of their nitrogen atoms, the compound I is capable of forming salts or adducts with inorganic or organic acids or with metal ions.

Examples of inorganic acids are hydrohalic acids, such as hydrogen fluoride, hydrogen chloride, hydrogen bromide und hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid groups), where the alkyl or aryl radicals may carry further substituents, for example p-toluenesulfonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminum, tin and lead and also of the elements of transition groups one to eight, in particular chromium, manganese, iron, cobalt, nickel, copper, zinc, and others. Particular preference is given to the metal ions of the elements of transition groups of the fourth period. The metals can be present in the various valencies that they can assume.

The preparation of the compounds of the formula I is known and described in detail in EP-A 0 094 564, EP-A 0 196 038 and EP-A 0 421 125.

An essential intermediate for the synthesis of the target compounds are α,β-disubstituted acroleins of type

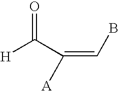

The synthesis of these compounds is described, inter alia, in DE3601927 by reaction of compounds of the type

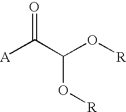

with phosphorus compounds of the Wittig or Horner-Emmons type with subsequent acidic cleavage of the acetal. R is preferably C₁-C₄-alkyl.

An alternative consists in the synthesis of α,β-disubstituted acrylic esters of the type:

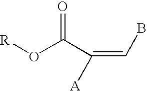

The reduction of acrolein can be carried out, for example, using metal hydrides, such as, for example, diisobutylaluminum hydride, at low temperatures. A further option is the reduction of the ester to the corresponding alcohol in order to obtain the desired oxidation state in a subsequent oxidation.

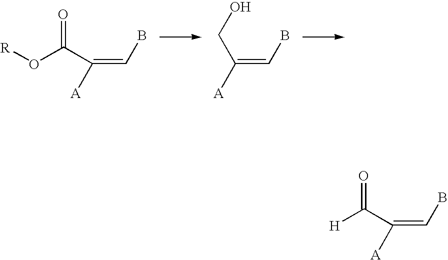

For this purpose, it is possible to use in particular aluminum hydrides, preferably lithium alanate (European Journal of Medicinal Chemistry, 40(6), 529-541; 2005) or dialkylaluminum hydrides, such as, for example, DIBAL-H (synlett, 18), 3182-3184; 2006). The appropriate oxidation reactions are known to the person skilled in the art, for example reactions according to Swern (Australian Journal of Chemistry, 57(6), 537-548; 2004), with hypervalent iodine compounds (Organic Letters, 5(17), 2989-2992; 2003), with chromium compounds such as, for example, pyridinium dichromate (Tetrahedron, 45(1), 239-58; 1989) or with manganese oxides, such as, for example MnO₂ (Journal of the American Chemical Society, 107(13), 3963-71; 1985).

The acrylic esters are available from glyoxalic esters by reaction with phosphorus compounds, for example compounds of the Horner-Emmons or Wittig type.

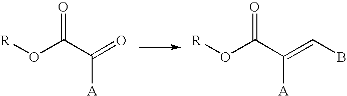

Such reactions are described, inter alia, in Tetrahedron, 46(13-14), 4951-94; 1990, Tetrahedron Letters, 47(16), 2675-2678; 2006, Synthesis, (12), 1797-1802, 2003, WO9929645 or Synthetic Communications, 20(12), 1781-91; 1990.

The synthesis of glyoxalic esters is described, inter alia, in Journal of Organic Chemistry, 52(22), 5026-30; 1987 by reaction of Grignard compounds (starting with commercial halogen compounds and magnesium) with oxalic esters.

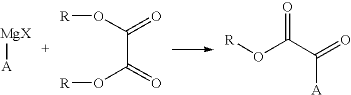

Suitable phosphorus compounds (of the Horner-Emmons type and Wittig type) can be prepared by known standard methods, for example from a compound of the following type:

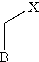

B is as defined above, X is a leaving group, such as, for example, a halide, preferably chlorine or bromine, very particularly preferably chlorine. The conversion of such halides into the desired Horner-Emmons or Wittig reagents can be carried out, for example, as described in DE2651968.

The alkyl halides are either commercially available or can be prepared by standard methods, for example by chlorination of the corresponding alcohol with a chlorinating agent, such as, for example, thionyl chloride (Tetrahedron: Asymmetry, 14(21), 3281-3283; 2003) or hydrochloric acid (Acta Chemica Scandinavica, 51(3, Suppl.), 392-402; 1997).

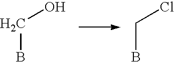

The preparation of corresponding bromides is described, for example, in Bioorganic & Medicinal Chemistry Letters, 16(1), 225-227; 2006, by reacting the methyl compound with a brominating agent, suitably N-bromosuccinimide.

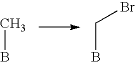

In the definitions of the symbols given in the formulae above, collective terms are used which are generally representative of the substituents below:

Halogen: fluorine, chlorine, bromine and iodine;

Alkyl and the alkyl moieties of composite groups such as, for example, alkylamino: saturated straight-chain or branched hydrocarbon radicals having preferably 1 to 4 carbon atoms, such as methyl, ethyl, propyl, 1-methylethyl, butyl, 1-methylpropyl, 2-methylpropyl and 1,1-dimethylethyl.

Haloalkyl: alkyl as mentioned above, where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₄)-haloalkyl, more preferably (C₁-C₂)-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

Alkoxy: an alkyl group as defined above which is attached via an oxygen, preferably having 1 to 4 carbon atoms. Examples of preferred alkoxy groups are: methoxy, ethoxy, n-propoxy, 1-methylethoxy, butoxy, 1-methylpropoxy, 2-methylpropoxy or 1,1-dimethylethoxy.

Haloalkoxy: alkoxy as defined above, where some or all of the hydrogen atoms in these groups are replaced by halogen atoms as described above under haloalkyl, in particular fluorine, chlorine or bromine. Examples of preferred haloalkoxy radicals are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

Alkylthio: alkyl as defined above which is attached via a sulfur atom.

5-membered heteroaryl: furyl, thienyl, pyrrolyl, pyrazolyl, imidazolyl, oxazolyl, isoxazolyl, thiazolyl and isothiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 3-oxazolyl, 4-oxazolyl, 5-oxazolyl, 2-thiazolyl, 4-thiazolyl, 5-thiazolyl, 2-imidazolyl and 4-imidazolyl. The heteroaryl can be attached via carbon or nitrogen, if present.

The novel compounds of the formula I contain chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

The compounds according to the invention may be present in various crystal modifications which may differ in their biological activity. They are likewise provided by the present invention.

In the compounds of the formula I according to the invention or in the compounds of the formula I used according to the invention, the following meanings of the substituents, in each case on their own or in combination, are particularly preferred. Here, the preferred substituents or preferred combinations of substituents apply, if appropriate, correspondingly to the precursors of the compounds according to the invention.

The substituent A or B is a 5-membered heteroaryl selected from the group consisting of thienyl, furyl, pyrrolyl, pyrazolyl, imidazolyl, oxazolyl, thiazolyl, isoxazolyl and isothiazolyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio.

According to one embodiment, the substituent A or B is selected from the group consisting of thienyl, furyl, pyrrolyl, pyrazolyl, imidazolyl and thiazolyl which is substituted by one to three of the abovementioned substituents.

According to a further embodiment, the substituent A or B is thienyl and pyrazolyl which is substituted by one to three of the abovementioned substituents.

According to another embodiment, the substituent A or B is thienyl which is substituted by one to three of the abovementioned substituents.

According to another embodiment, the substituent A or B is furyl which is substituted by one to three of the abovementioned substituents.

According to another embodiment, the substituent A or B is pyrrolyl which is substituted by one to three of the abovementioned substituents.

According to another embodiment, the substituent A or B is pyrazolyl which is substituted by one to three of the abovementioned substituents.

According to another embodiment, the substituent A or B is imidazolyl which is substituted by one to three of the abovementioned substituents.

According to another embodiment, the substituent A or B is oxazolyl which is substituted by one to three of the abovementioned substituents.

According to another embodiment, the substituent A or B is thiazolyl which is substituted by one to three of the abovementioned substituents.

According to another embodiment, the substituent A or B is isoxazolyl which is substituted by one to three of the abovementioned substituents.

According to another embodiment, the substituent A or B is isothiazolyl which is substituted by one to three of the abovementioned substituents.

According to a further embodiment, the 5-membered heteroaryl is substituted by one to three of the following substituents: halogen, C₁-C₄-alkyl, C₁-C₄-alkoxy, C₁-C₄-haloalkyl or C₁-C₄-haloalkoxy.

According to another embodiment, the 5-membered heteroaryl is substituted by one to three of the following substituents: halogen, C₁-C₄-alkyl or C₁-C₄-alkoxy.

According to a preferred embodiment, the 5-membered heteroaryl is substituted by one to three of the following substituents: F, Cl, methyl or methoxy.

According to a further preferred embodiment, the 5-membered heteroaryl is substituted by one to three F or Cl substituents.

The respective other substituent A or B which is different to 5-membered heteroaryl is unsubstituted phenyl or is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio.

According to a further embodiment, the respective other substituent A or B which is different from 5-membered heteroaryl is phenyl which is substituted by one to three of the following substituents: halogen, C₁-C₄-alkyl, C₁-C₄-alkoxy, C₁-C₄-haloalkyl or C₁-C₄-haloalkoxy.

According to a further embodiment, the respective other substituent A or B which is different from 5-membered heteroaryl is phenyl which is substituted by one to three of the following substituents: halogen, C₁-C₄-alkyl or C₁-C₄-alkoxy.

According to a further embodiment, the respective other substituent A or B which is different from 5-membered heteroaryl is phenyl which is substituted by one to three halogen substituents.

According to a further embodiment, the respective other substituent A or B which is different from 5-membered heteroaryl is phenyl which is substituted by one to three of the following substituents: F, Cl, methyl or methoxy.

According to a further embodiment, the respective other substituent A or B which is different from 5-membered heteroaryl is phenyl which is substituted by one to three F or Cl substituents.

In a preferred embodiment, B is phenyl which is substituted by one to three halogens.

In particular with a view to their use, preference is given to the compounds I according to the invention compiled in Tables 5 to 70 below. The groups mentioned for a substituent in the tables are furthermore per se, independently of the combination in which they are mentioned, a particularly preferred embodiment of the substituent in question.

TABLE 1

| Row | Substituent A |
|---|---|
| 1-1 | 2-chlorothien-3-yl |
| 1-2 | 4-chlorothien-3-yl |
| 1-3 | 5-chlorothien-3-yl |
| 1-4 | 2,5-dichlorothien-3-yl |
| 1-5 | 2,4,5-trichlorothien-3-yl |
| 1-6 | 2-bromothien-3-yl |
| 1-7 | 4-bromothien-3-yl |
| 1-8 | 5-bromothien-3-yl |
| 1-9 | 2,5-dibromothien-3-yl |
| 1-10 | 2,4,5-tribromothien-3-yl |
| 1-11 | 3-bromofur-2-yl |
| 1-12 | 4-bromofur-2-yl |
| 1-13 | 5-bromofur-2-yl |
| 1-14 | 4,5-dibromofur-2-yl |
| 1-15 | 4-chlorofur-2-yl |
| 1-16 | 5-chlorofur-2-yl |
| 1-17 | 3,4-dichlorofur-2-yl |
| 1-18 | 2-chlorothiazol-5-yl |
| 1-19 | 4-chlorothiazol-5-yl |
| 1-20 | 2,4-dichlorothiazol-5-yl |
| 1-21 | 2-bromothiazol-5-yl |
| 1-22 | 2-chlorothiazol-4-yl |
| 1-23 | 2-bromothiazol-4-yl |
| 1-24 | 1-methylpyrazol-4-yl |
| 1-25 | 1,3-dimethylpyrazol-4-yl |
| 1-26 | 1,5-dimethylpyrazol-4-yl |
| 1-27 | 1,3,5-trimethylpyrazol-4-yl |
| 1-28 | 1-methylpyrazol-3-yl |
| 1-29 | 1-methylimidazol-4-yl |
| 1-30 | 1,5-dimethylimidazol-4-yl |
| 1-31 | 1-methylpyrazol-5-yl |
| 1-32 | 1,2-dimethylimidazol-5-yl |
| 1-33 | 1,4-dimethylimidazol-5-yl |

TABLE 2

| Row | Substituent B |
|---|---|
| 2-1 | phenyl |
| 2-2 | 2-methylphenyl |
| 2-3 | 3-methylphenyl |
| 2-4 | 4-methylphenyl |
| 2-5 | 2-methoxyphenyl |
| 2-6 | 3-methoxyphenyl |
| 2-7 | 4-methoxyphenyl |
| 2-8 | 2-chlorophenyl |
| 2-9 | 3-chlorophenyl |
| 2-10 | 4-chlorophenyl |
| 2-11 | 2-fluorophenyl |
| 2-12 | 3-fluorophenyl |
| 2-13 | 4-fluorophenyl |
| 2-14 | 2-chloro-3-methoxyphenyl |
| 2-15 | 2-chloro-4-methoxyphenyl |
| 2-16 | 2,3-dichlorophenyl |
| 2-17 | 2,4-dichlorophenyl |
| 2-18 | 3,4-dichlorophenyl |
| 2-19 | 2,3-difluorophenyl |
| 2-20 | 2,4-difluorophenyl |
| 2-21 | 2-chloro-3-fluorophenyl |
| 2-22 | 2-chloro-4-fluorophenyl |
| 2-23 | 2-pyridyl |
| 2-24 | 3-pyridyl |
| 2-25 | 4-pyridyl |
| 2-26 | 2-thienyl |
| 2-27 | 3-thienyl |
| 2-28 | thiazol-4-yl |
| 2-29 | thiazol-5-yl |
| 2-30 | oxazol-4-yl |
| 2-31 | oxazol-5-yl |
| 2-32 | 2-furyl |

TABLE 3

| Row | Substituent B |
|---|---|
| 3-1 | 2-chlorothien-3-yl |
| 3-2 | 4-chlorothien-3-yl |
| 3-3 | 5-chlorothien-3-yl |
| 3-4 | 2,5-dichlorothien-3-yl |
| 3-5 | 2,4,5-trichlorothien-3-yl |
| 3-6 | 2-bromothien-3-yl |
| 3-7 | 4-bromothien-3-yl |
| 3-8 | 5-bromothien-3-yl |
| 3-9 | 2,5-dibromothien-3-yl |
| 3-10 | 2,4,5-tribromothien-3-yl |
| 3-11 | 3-bromofur-2-yl |
| 3-12 | 4-bromofur-2-yl |
| 3-13 | 5-bromofur-2-yl |
| 3-14 | 4,5-dibromofur-2-yl |
| 3-15 | 4-chlorofur-2-yl |
| 3-16 | 5-chlorofur-2-yl |
| 3-17 | 3,4-dichlorofur-2-yl |
| 3-18 | 2-chlorothiazol-5-yl |
| 3-19 | 4-chlorothiazol-5-yl |
| 3-20 | 2,4-dichlorothiazol-5-yl |
| 3-21 | 2-bromothiazol-5-yl |
| 3-22 | 2-chlorothiazol-4-yl |
| 3-23 | 2-bromothiazol-4-yl |
| 3-24 | 1-methylpyrazol-4-yl |
| 3-25 | 1,3-dimethylpyrazol-4-yl |
| 3-26 | 1,5-dimethylpyrazol-4-yl |
| 3-27 | 1,3,5-trimethylpyrazol-4-yl |
| 3-28 | 1-methylpyrazol-3-yl |
| 3-29 | 1-methylimidazol-4-yl |
| 3-30 | 1,5-dimethylimidazol-4-yl |
| 3-31 | 1-methylpyrazol-5-yl |
| 3-32 | 1,2-dimethylimidazol-5-yl |
| 3-33 | 1,4-dimethylimidazol-5-yl |

TABLE 4

| Row | Substituent A |
|---|---|
| 4-1 | phenyl |
| 4-2 | 2-methylphenyl |
| 4-3 | 3-methylphenyl |
| 4-4 | 4-methylphenyl |
| 4-5 | 2-methoxyphenyl |
| 4-6 | 3-methoxyphenyl |
| 4-7 | 4-methoxyphenyl |
| 4-8 | 2-chlorophenyl |
| 4-9 | 3-chlorophenyl |
| 4-10 | 4-chlorophenyl |
| 4-11 | 2-fluorophenyl |
| 4-12 | 3-fluorophenyl |
| 4-13 | 4-fluorophenyl |
| 4-14 | 2-chloro-3-methoxyphenyl |
| 4-15 | 2-chloro-4-methoxyphenyl |
| 4-16 | 2,3-dichlorophenyl |
| 4-17 | 2,4-dichlorophenyl |
| 4-18 | 3,4-dichlorophenyl |
| 4-19 | 2,3-difluorophenyl |
| 4-20 | 2,4-difluorophenyl |
| 4-21 | 2-chloro-3-fluorophenyl |
| 4-22 | 2-chloro-4-fluorophenyl |
| 4-23 | 2-pyridyl |
| 4-24 | 3-pyridyl |
| 4-25 | 4-pyridyl |
| 4-26 | 2-thienyl |
| 4-27 | 3-thienyl |
| 4-28 | thiazol-4-yl |
| 4-29 | thiazol-5-yl |
| 4-30 | oxazol-4-yl |
| 4-31 | oxazol-5-yl |
| 4-32 | 2-furyl |

### Table 5

Compounds of the formula I in which A is substituent 1-1 of Table 1 and B corresponds in each case to a substituent of a row of Table 2.

### Table 6

Compounds of the formula I in which A is substituent 1-2 of Table 1and B corresponds in each case to a substituent of a row of Table 2.

### Table 7

Compounds of the formula I in which A is substituent 1-3 of Table 1 and B corresponds in each case to a substituent of a row of Table 2.

### Table 8

Compounds of the formula I in which A is substituent 1-4 of Table 1 and B corresponds in each case to a substituent of a row of Table 2.

### Table 9

Compounds of the formula I in which A is substituent 1-5 of Table 1 and B corresponds in each case to a substituent of a row of Table 2.

### Table 10

Compounds of the formula I in which A is substituent 1-6 of Table 1 and B corresponds in each case to a substituent of a row of Table 2.

### Table 11

Compounds of the formula I in which A is substituent 1-7 of Table 1 and B corresponds in each case to a substituent of a row of Table 2.

### Table 12

Compounds of the formula I in which A is substituent 1-8 of Table 1 and B corresponds in each case to a substituent of a row of Table 2.

### Table 13

Compounds of the formula I in which A is substituent 1-9 of Table 1 and B corresponds in each case to a substituent of a row of Table 2.

### Table 14

Compounds of the formula I in which A is substituent 1-10 of Table 1 and B corresponds in each case to a substituent of a row of Table 2.

### Table 15

Compounds of the formula I in which A is substituent 1-11 of Table 1 and B corresponds in each case to a substituent of a row of Table 2.

### Table 16

Compounds of the formula I in which A is substituent 1-12 of Table 1 and B corresponds in each case to a substituent of a row of Table 2.

### Table 17

Compounds of the formula I in which A is substituent 1-13 of Table 1 and B corresponds in each case to a substituent of a row of Table 2.

### Table 18

Compounds of the formula I in which A is substituent 1-14 of Table 1 and B corresponds in each case to a substituent of a row of Table 2.

### Table 19

Compounds of the formula I in which A is substituent 1-15 of Table 1 and B corresponds in each case to a substituent of a row of Table 2.

### Table 20

Compounds of the formula I in which A is substituent 1-16 of Table 1 and B corresponds in each case to a substituent of a row of Table 2.

### Table 21

Compounds of the formula I in which A is substituent 1-17 of Table 1 and B corresponds in each case to a substituent of a row of Table 2.

### Table 22

Compounds of the formula I in which A is substituent 1-18 of Table 1 and B corresponds in each case to a substituent of a row of Table 2.

### Table 23

Compounds of the formula I in which A is substituent 1-19 of Table 1 and B corresponds in each case to a substituent of a row of Table 2.

### Table 24

Compounds of the formula I in which A is substituent 1-20 of Table 1 and B corresponds in each case to a substituent of a row of Table 2.

### Table 25

Compounds of the formula I in which A is substituent 1-21 of Table 1 and B corresponds in each case to a substituent of a row of Table 2.

### Table 26

Compounds of the formula I in which A is substituent 1-22 of Table 1 and B corresponds in each case to a substituent of a row of Table 2.

### Table 27

Compounds of the formula I in which A is substituent 1-23 of Table 1 and B corresponds in each case to a substituent of a row of Table 2.

### Table 28

Compounds of the formula I in which A is substituent 1-24 of Table 1 and B corresponds in each case to a substituent of a row of Table 2.

### Table 29

Compounds of the formula I in which A is substituent 1-25 of Table 1 and B corresponds in each case to a substituent of a row of Table 2.

### Table 30

Compounds of the formula I in which A is substituent 1-26 of Table 1 and B corresponds in each case to a substituent of a row of Table 2.

### Table 31

Compounds of the formula I in which A is substituent 1-27 of Table 1 and B corresponds in each case to a substituent of a row of Table 2.

### Table 32

Compounds of the formula I in which A is substituent 1-28 of Table 1 and B corresponds in each case to a substituent of a row of Table 2.

### Table 33

Compounds of the formula I in which A is substituent 1-29 of Table 1 and B corresponds in each case to a substituent of a row of Table 2.

### Table 34

Compounds of the formula I in which A is substituent 1-30 of Table 1 and B corresponds in each case to a substituent of a row of Table 2.

### Table 35

Compounds of the formula I in which A is substituent 1-31 of Table 1 and B corresponds in each case to a substituent of a row of Table 2.

### Table 36

Compounds of the formula I in which A is substituent 1-32 of Table 1 and B corresponds in each case to a substituent of a row of Table 2.

### Table 37

Compounds of the formula I in which A is substituent 1-33 of Table 1 and B corresponds in each case to a substituent of a row of Table 2.

### Table 38

Compounds of the formula I in which B is substituent 3-1 of Table 3 and A corresponds in each case to a substituent of a row of Table 4.

### Table 39

Compounds of the formula I in which B is substftuent 3-2 of Table 3 and A corresponds in each case to a substituent of a row of Table 4.

### Table 40

Compounds of the formula I in which B is substituent 3-3 of Table 3 and A corresponds in each case to a substituent of a row of Table 4.

### Table 41

Compounds of the formula I in which B is substituent 3-4 of Table 3 and A corresponds in each case to a substituent of a row of Table 4.

### Table 42

Compounds of the formula I in which B is substituent 3-5 of Table 3 and A corresponds in each case to a substituent of a row of Table 4.

### Table 43

Compounds of the formula I in which B is substituent 3-6 of Table 3 and A corresponds in each case to a substituent of a row of Table 4.

### Table 44

Compounds of the formula I in which B is substituent 3-7 of Table 3 and A corresponds in each case to a substituent of a row of Table 4.

### Table 45

Compounds of the formula I in which B is substituent 3-8 of Table 3 and A corresponds in each case to a substituent of a row of Table 4.

### Table 46

Compounds of the formula I in which B is substituent 3-9 of Table 3 and A corresponds in each case to a substituent of a row of Table 4.

### Table 47

Compounds of the formula I in which B is substituent 3-10 of Table 3 and A corresponds in each case to a substituent of a row of Table 4.

### Table 48

Compounds of the formula I in which B is substituent 3-11 of Table 3 and A corresponds in each case to a substituent of a row of Table 4.

### Table 49

Compounds of the formula I in which B is substituent 3-12 of Table 3 and A corresponds in each case to a substituent of a row of Table 4.

### Table 50

Compounds of the formula I in which B is substituent 3-13 of Table 3 and A corresponds in each case to a substituent of a row of Table 4.

### Table 51

Compounds of the formula I in which B is substituent 3-14 of Table 3 and A corresponds in each case to a substituent of a row of Table 4.

### Table 52

Compounds of the formula I in which B is substituent 3-15 of Table 3 and A corresponds in each case to a substituent of a row of Table 4.

### Table 53

Compounds of the formula I in which B is substituent 3-16 of Table 3 and A corresponds in each case to a substituent of a row of Table 4.

### Table 54

Compounds of the formula I in which B is substituent 3-17 of Table 3 and A corresponds in each case to a substituent of a row of Table 4.

### Table 55

Compounds of the formula I in which B is substituent 3-18 of Table 3 and A corresponds in each case to a substituent of a row of Table 4.

### Table 56

Compounds of the formula I in which B is substituent 3-19 of Table 3 and A corresponds in each case to a substituent of a row of Table 4.

### Table 57

Compounds of the formula I in which B is substituent 3-20 of Table 3 and A corresponds in each case to a substituent of a row of Table 4.

### Table 58

Compounds of the formula I in which B is substituent 3-21 of Table 3 and A corresponds in each case to a substituent of a row of Table 4.

### Table 59

Compounds of the formula I in which B is substituent 3-22 of Table 3 and A corresponds in each case to a substituent of a row of Table 4.

### Table 60

Compounds of the formula I in which B is substituent 3-23 of Table 3 and A corresponds in each case to a substituent of a row of Table 4.

### Table 61

Compounds of the formula I in which B is substituent 3-24 of Table 3 and A corresponds in each case to a substituent of a row of Table 4.

### Table 62

Compounds of the formula I in which B is substituent 3-25 of Table 3 and A corresponds in each case to a substituent of a row of Table 4.

### Table 63

Compounds of the formula I in which B is substituent 3-26 of Table 3 and A corresponds in each case to a substituent of a row of Table 4.

### Table 64

Compounds of the formula I in which B is substituent 3-27 of Table 3 and A corresponds in each case to a substituent of a row of Table 4.

### Table 65

Compounds of the formula I in which B is substituent 3-28 of Table 3 and A corresponds in each case to a substituent of a row of Table 4.

### Table 66

Compounds of the formula I in which B is substituent 3-29 of Table 3 and A corresponds in each case to a substituent of a row of Table 4.

### Table 67

Compounds of the formula I in which B is substituent 3-30 of Table 3 and A corresponds in each case to a substituent of a row of Table 4.

### Table 68

Compounds of the formula I in which B is substituent 3-31 of Table 3 and A corresponds in each case to a substituent of a row of Table 4.

### Table 69

Compounds of the formula I in which B is substituent 3-32 of Table 3 and A corresponds in each case to a substituent of a row of Table 4.

### Table 70

Compounds of the formula I in which B is substituent 3-33 of Table 3 and A corresponds in each case to a substituent of a row of Table 4.

The compounds I are suitable as fungicides. They are distinguished by an excellent activity against a broad spectrum of phytopathogenic fungi from the class of the Ascomycetes, Deuteromycetes, Oomycetes and Basidiomycetes, in particular from the class of the Oomycetes. Some of them are systemically effective and can be used in crop protection as foliar fungicides, as fungicides for seed dressing and as soil fungicides.

They are particularly important in the control of a multitude of fungi on various crop plants, such as wheat, rye, barley, oats, rice, corn, grass, bananas, cotton, soya, coffee, sugar cane, vines, fruit and ornamental plants, and vegetable plants, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example, Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumber plants;
Fusarium and Verticillium species on various plants, such as, for example,
F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onion;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apple;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apple.

They are particularly suitable for controlling harmful fungi from the class of the Peronosporomycetes (syn. Oomycetes), such as Peronospora species, Phytophthora species, Plasmopara viticola, Pseudoperonospora species and Pythium species.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Scierophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds or materials to be protected against fungal attack or the soil with a fungicidally effective amount of the active compounds. Application can be both before and after the infection of the materials, plants or seeds by the fungi.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rates depend on the kind of application area and on the desired effect.

Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

### 2. Products to be Applied Undiluted

### I Dusts (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

### K ULV Solutions (UL)

10 parts by weight of the active compounds are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention in the application form as fungicides can also be present together with other active compounds, for example with herbicides, insecticides, growth regulators, fungicides or else with fertilizers. When mixing the compounds I or the compositions comprising them with one or more further active compounds, in particular fungicides, it is in many cases possible, for example, to widen the activity spectrum or to prevent the development of resistance. In many cases, synergistic effects are obtained.

The present invention furthermore provides a combination of at least one azolylmethyloxirane of the formula I, in particular an azolylmethyloxirane disclosed in the present description as being preferred, and/or an agriculturally acceptable salt thereof and at least one further fungicidal, insecticidal, herbicidal and/or growth-regulating active compound, it being possible for a synergistic effect to occur.

The present invention also provides a pesticidal composition which comprises at least one compound of the formula I, in particular a compound of the formula I described in the present description as being preferred, and/or an agriculturally acceptable acid addition salt or metal salt thereof and at least one solid or liquid carrier. Such a pesticidal composition may comprise at least one further fungicidally, insecticidally and/or herbicidally active compound, it also being possible for a synergistic effect to occur.

The following list L of fungicides with which the compounds according to the invention can be applied together is meant to illustrate the possible combinations, but not to limit them:

### List L:

### Strobilurins

azoxystrobin, dimoxystrobin, enestroburin, fluoxastrobin, kresoxim-methyl, methominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxymethylene)phenyl)-3-methoxyacrylate, methyl 3-methoxy-2-(2-(N-(4-methoxyphenyl)cyclopropanecarboximidoylsulfanylmethyl)phenyl)acrylate;

### Carboxamides

carboxanilides: benalaxyl, benalaxyl-M, benodanil, bixafen, boscalid, carboxin, fenfuram, fenhexamid, flutolanil, furametpyr, isotianil, kiralaxyl, mepronil, metalaxyl, ofurace, oxadixyl, oxycarboxin, penthiopyrad, tecloftalam, thifluzamide, tiadinil, 2-amino-4-methylthiazole-5-carboxanilide, 2-chloro-N-(1,1,3-trimethylindan-4-yl)nicotinamide, N-(3′,4′-dichloro-5-fluorobiphenyl-2-yl)-3-difluoromethyl-1-methyl-1H-pyrazole-4-carboxamide, N-[2-(1,3-dimethylbutyl)phenyl]-5-fluoro-1,3-dimethyl-1H-pyrazole-4-carboxamide, N-(4′-chloro-3′,5-difluorobiphenyl-2-yl)-3-difluoromethyl-1-methyl-1H-pyrazole-4-carboxamide, N-(4′-chloro-3′,5-difluorobiphenyl-2-yl)-3-trifluoromethyl-1-methyl-1H-pyrazole-4-carboxamide, N-(3′,4′-dichloro-5-fluorobiphenyl-2-yl)-3-trifluoromethyl-1-methyl-1H-pyrazole-4-carboxamide, N-(3′,5-difluoro-4′-methylbiphenyl-2-yl)-3-difluoromethyl-1-methyl-1H-pyrazole-4-carboxamide, N-(3′,5-difluoro-4′-methylbiphenyl-2-yl)-3-trifluoromethyl-1-methyl-1H-pyrazole-4-carboxamide, N-(2-bicyclopropyl-2-ylphenyl)-3-difluoromethyl-1-methyl-1H-pyrazole-4-carboxamide, N-(cis-2-bicyclopropyl-2-ylphenyl)-3-difluoromethyl-1-methyl-1H-pyrazole-4-carboxamide, N-(trans-2-bicyclopropyl-2-ylphenyl)-3-difluoromethyl-1-methyl-1H-pyrazole-4-carboxamide,
carboxylic acid morpholides: dimethomorph, flumorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;

### Azoles

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)cycloheptanol;
imidazoles: cyazofamid, imazalil, imazalil-sulfate, pefurazoate, prochloraz, triflumizole;
benzimidazoles: benomyl, carbendazim, fuberidazole, thiabendazole;
others: ethaboxam, etridiazole, hymexazole, 1-(4-chlorophenyl)-1-(propyn-2-yloxy)-3-(4-(3,4-dimethoxyphenyl)isoxazol-5-yl)propan-2-one;

### Nitrogenous Heterocyclyl Compounds

pyridines: fluazinam, pyrifenox, 3-[5-(4-chlorophenyl)-2,3-dimethylisoxazolidin-3-yl]-pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
pyrroles: fludioxonil, fenpiclonil;
morpholines: aldimorph, dodemorph, dodemorph-acetate, fenpropimorph, tridemorph;
dicarboximides: fluoroimide, iprodione, procymidone, vinclozolin;
others: acibenzolar-5-methyl, amisulbrom, anilazine, blasticidin-S, captafol, captan, quinomethionate, dazomet, debacarb, diclomezine, difenzoquat, difenzoquat-methyl sulfate, famoxadone, fenamidone, fenoxanil, fenpropidin, folpet, octhilinone, oxolinic acid, piperalin, probenazole, proquinazid, pyroquilon, quinoxyfen, triazoxide, tricyclazole, triforine, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one;

### Carbamates and Dithiocarbamates

thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, (4-fluorophenyl) N-(1-(1-(4-cyanophenyl)ethanesulfonyl)but-2-yl)carbamate;

### Other Fungicides

guanidines: dodine, dodine-free base, guazatine, guazatine-acetate, iminoctadine, iminoctadine-triacetate, iminoctadine-tris(albesilate);
antibiotics: kasugamycin, kasugamycin-hydrochloride-hydrate, polyoxins, streptomycin, validamycin A;
nitrophenyl derivatives:
   binapacryl, dicloran, dinobuton, dinocap, nitrothal-isopropyl, tecnazen;
organometallic compounds: fentin salts such as, for example, fentin-acetate, fentinchloride, fentin-hydroxide;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl;
organochlorine compounds: chlorothalonil, dichlofluanid, dichlorophen, flusulfamide, hexachlorobenzene, pencycuron, pentachlorophenol and salts thereof, phthalide, quintozene, thiophanate-methyl, tolylfluanid, N-(4-chloro-2-nitrophenyl)-N-ethyl-4-methylbenzenesulfonamide;
inorganic active compounds: phosphorous acid and salts thereof, sulfur, Bordeaux mixture, copper salts such as, for example, copper acetate, copper hydroxide, copper oxychloride, basic copper sulfate;
others: biphenyl, bronopol, cyflufenamid, cymoxanil, diphenylamin, metrafenone, mildiomycin, oxine-copper, prohexadione-calcium, spiroxamine, tolylfluanid, N-(cyclopropylmethoxyimino-(6-difluoromethoxy-2,3-difluorophenyl)methyl)-2-phenyl acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethylsilanylpropoxy)phenyl)-N-ethyl-N-methylformamidine.

The present invention further relates, accordingly, to the compositions that are listed in table B, each line of table B corresponding to a fungicidal composition comprising a compound of the formula I (component 1), which is preferably one of the compounds described herein as being preferred, and comprising the further active compound indicated in each case in the line in question (component 2). According to one embodiment of the invention, component 1 in each line of table B is in each case one of the compounds of the formula I that are specifically individualized in tables 1 to xxxx.

TABLE B

| Line | Component 1 | Component 2 |
|---|---|---|
| B-1 | a compound of the formula I | azoxystrobin |
| B-2 | a compound of the formula I | dimoxystrobin |
| B-3 | a compound of the formula I | enestroburin |
| B-4 | a compound of the formula I | fluoxastrobin |
| B-5 | a compound of the formula I | kresoxim-methyl |
| B-6 | a compound of the formula I | metominostrobin |
| B-7 | a compound of the formula I | orysastrobin |
| B-8 | a compound of the formula I | picoxystrobin |
| B-9 | a compound of the formula I | pyraclostrobin |
| B-10 | a compound of the formula I | pyribencarb |
| B-11 | a compound of the formula I | trifloxystrobin |
| B-12 | a compound of the formula I | 2-(2-(6-(3-chloro-2-methylphenoxy)- |
| | | 5-fluoropyrimidin-4-yloxy)phenyl)- |
| | | 2-methoxyimino-N-methylacetamide |
| B-13 | a compound of the formula I | 2-(ortho-((2,5-dimethylphenyloxy- |
| | | methylene)phenyl)-3-methoxyacrylic acid |
| | | methyl ester |
| B-14 | a compound of the formula I | 3-methoxy-2-(2-(N-(4-methoxyphenyl)- |
| | | cyclopropanecarboximidoylsulfanyl- |
| | | methyl)phenyl)acrylic acid methyl ester |
| B-15 | a compound of the formula I | benalaxyl |
| B-16 | a compound of the formula I | benalaxyl-M |
| B-17 | a compound of the formula I | benodanil |
| B-18 | a compound of the formula I | bixafen |
| B-19 | a compound of the formula I | boscalid |
| B-20 | a compound of the formula I | carboxin |
| B-21 | a compound of the formula I | fenfuram |
| B-22 | a compound of the formula I | fenhexamid |
| B-23 | a compound of the formula I | flutolanil |
| B-24 | a compound of the formula I | furametpyr |
| B-25 | a compound of the formula I | isotianil |
| B-26 | a compound of the formula I | kiralaxyl |
| B-27 | a compound of the formula I | mepronil |
| B-28 | a compound of the formula I | metalaxyl |
| B-29 | a compound of the formula I | ofurace |
| B-30 | a compound of the formula I | oxadixyl |
| B-31 | a compound of the formula I | oxycarboxin |
| B-32 | a compound of the formula I | penthiopyrad |
| B-33 | a compound of the formula I | thifluzamide |
| B-34 | a compound of the formula I | tecloftalam |
| B-35 | a compound of the formula I | tiadinil |
| B-36 | a compound of the formula I | 2-amino-4-methylthiazole-5-carboxanilide |
| B-37 | a compound of the formula I | 2-chloro-N-(1,1,3-trimethylindan-4-yl)- |
| | | nicotinamide |
| B-38 | a compound of the formula I | N-(3′,4′-dichloro-5-fluorobiphenyl-2-yl)- |
| | | 3-difluoromethyl-1-methyl-1H-pyrazole- |
| | | 4-carboxamide |
| B-39 | a compound of the formula I | 5-fluoro-1,3-dimethyl-1H-pyrazole- |
| | | 4-carboxylic acid [2-(1,3-dimethylbutyl)- |
| | | phenyl]amide |
| B-40 | a compound of the formula I | N-(4′-chloro-3′,5-difluorobiphenyl-2-yl)- |
| | | 3-difluoromethyl-1-methyl-1H-pyrazole- |
| | | 4-carboxamide |
| B-41 | a compound of the formula I | N-(4′-chloro-3′,5-difluorobiphenyl-2-yl)- |
| | | 3-trifluoromethyl-1-methyl-1H-pyrazole- |
| | | 4-carboxamide |
| B-42 | a compound of the formula I | N-(3′,4′-dichloro-5-fluorobiphenyl-2-yl)- |
| | | 3-trifluoromethyl-1-methyl-1H-pyrazole- |
| | | 4-carboxamide |
| B-43 | a compound of the formula I | N-(3′,5-difluoro-4′-methylbiphenyl-2-yl)- |
| | | 3-difluoromethyl-1-methyl-1H-pyrazole- |
| | | 4-carboxamide |
| B-44 | a compound of the formula I | N-(3′,5-difluoro-4′-methylbiphenyl-2-yl)- |
| | | 3-trifluoromethyl-1-methyl-1H-pyrazole- |
| | | 4-carboxamide |
| B-45 | a compound of the formula I | N-(2-bicyclopropyl-2-yl-phenyl)-3-difluoro- |
| | | methyl-1-methyl-1H-pyrazole-4-carboxamide |
| B-46 | a compound of the formula I | N-(cis-2-bicyclopropyl-2-yl-phenyl)- |
| | | 3-difluoromethyl-1-methyl-1H-pyrazole- |
| | | 4-carboxamide |
| B-47 | a compound of the formula I | N-(trans-2-bicyclopropyl-2-yl-phenyl)-3- |
| | | difluoromethyl-1-methyl-1H-pyrazole- |
| | | 4-carboxamide |
| B-48 | a compound of the formula I | dimethomorph |
| B-49 | a compound of the formula I | flumorph |
| B-50 | a compound of the formula I | flumetover |
| B-51 | a compound of the formula I | fluopicolide (picobenzamid) |
| B-52 | a compound of the formula I | fluopyram |
| B-53 | a compound of the formula I | zoxamide |
| B-54 | a compound of the formula I | N-(3-ethyl-3,5,5-trimethylcyclohexyl)- |
| | | 3-formylamino-2-hydroxybenzamide |
| B-55 | a compound of the formula I | carpropamid |
| B-56 | a compound of the formula I | diclocymet |
| B-57 | a compound of the formula I | mandipropamid |
| B-58 | a compound of the formula I | oxytetracyclin |
| B-59 | a compound of the formula I | silthiofam |
| B-60 | a compound of the formula I | N-(6-methoxypyridin-3-yl)cyclopropane- |
| | | carboxamide |
| B-61 | a compound of the formula I | azaconazole |
| B-62 | a compound of the formula I | bitertanol |
| B-63 | a compound of the formula I | bromuconazole |
| B-64 | a compound of the formula I | cyproconazole |
| B-65 | a compound of the formula I | difenoconazole |
| B-66 | a compound of the formula I | diniconazole |
| B-67 | a compound of the formula I | diniconazole-M |
| B-68 | a compound of the formula I | enilconazole |
| B-69 | a compound of the formula I | epoxiconazole |
| B-70 | a compound of the formula I | fenbuconazole |
| B-71 | a compound of the formula I | flusilazole |
| B-72 | a compound of the formula I | fluquinconazole |
| B-73 | a compound of the formula I | flutriafol |
| B-74 | a compound of the formula I | hexaconazol |
| B-75 | a compound of the formula I | imibenconazole |
| B-76 | a compound of the formula I | ipconazole |
| B-77 | a compound of the formula I | metconazol |
| B-78 | a compound of the formula I | myclobutanil |
| B-79 | a compound of the formula I | oxpoconazol |
| B-80 | a compound of the formula I | paclobutrazol |
| B-81 | a compound of the formula I | penconazole |
| B-82 | a compound of the formula I | propiconazole |
| B-83 | a compound of the formula I | prothioconazole |
| B-84 | a compound of the formula I | simeconazole |
| B-85 | a compound of the formula I | tebuconazole |
| B-86 | a compound of the formula I | tetraconazole |
| B-87 | a compound of the formula I | triadimenol |
| B-88 | a compound of the formula I | triadimefon |
| B-89 | a compound of the formula I | triticonazole |
| B-90 | a compound of the formula I | uniconazol |
| B-91 | a compound of the formula I | 1-(4-chlorophenyl)-2-([1,2,4]triazol-1-yl)- |
| | | cycloheptanol |
| B-92 | a compound of the formula I | cyazofamid |
| B-93 | a compound of the formula I | imazalil |
| B-94 | a compound of the formula I | imazalil-sulfate |
| B-95 | a compound of the formula I | pefurazoate |
| B-96 | a compound of the formula I | prochloraz |
| B-97 | a compound of the formula I | triflumizole |
| B-98 | a compound of the formula I | benomyl |
| B-99 | a compound of the formula I | carbendazim |
| B-100 | a compound of the formula I | fuberidazole |
| B-101 | a compound of the formula I | thiabendazole |
| B-102 | a compound of the formula I | ethaboxam |
| B-103 | a compound of the formula I | etridiazole |
| B-104 | a compound of the formula I | hymexazole |
| B-105 | a compound of the formula I | fluazinam |
| B-106 | a compound of the formula I | pyrifenox |
| B-107 | a compound of the formula I | 1-(4-chlorophenyl)-1-(propyn-2-yloxy)- |
| | | 3-(4-(3,4-dimethoxyphenyl)isoxazol-5-yl)- |
| | | propan-2-one |
| B-108 | a compound of the formula I | 3-[5-(4-chlorophenyl)-2,3-dimethyl- |
| | | isoxazolidin-3-yl]pyridine |
| B-109 | a compound of the formula I | 2,3,5,6-tetrachloro-4-methanesulfonyl- |
| | | pyridine |
| B-110 | a compound of the formula I | 3,4,5-trichloropyridine-2,6-dicarbonitrile |
| B-111 | a compound of the formula I | N-(1-(5-bromo-3-chloropyridin-2-yl)ethyl)-2,4- |
| | | dichloronicotinamide |
| B-112 | a compound of the formula I | N-((5-bromo-3-chloropyridin-2-yl)methyl)-2,4- |
| | | dichloronicotinamide |
| B-113 | a compound of the formula I | bupirimate |
| B-114 | a compound of the formula I | cyprodinil |
| B-115 | a compound of the formula I | diflumetorim |
| B-116 | a compound of the formula I | ferimzone |
| B-117 | a compound of the formula I | fenarimol |
| B-118 | a compound of the formula I | mepanipyrim |
| B-119 | a compound of the formula I | nitrapyrin |
| B-120 | a compound of the formula I | nuarimol |
| B-121 | a compound of the formula I | pyrimethanil |
| B-122 | a compound of the formula I | fludioxonil |
| B-123 | a compound of the formula I | fenpiclonil |
| B-124 | a compound of the formula I | aldimorph |
| B-125 | a compound of the formula I | dodemorph |
| B-126 | a compound of the formula I | dodemorph acetate |
| B-127 | a compound of the formula I | fenpropimorph |
| B-128 | a compound of the formula I | tridemorph |
| B-129 | a compound of the formula I | fluoroimid |
| B-130 | a compound of the formula I | iprodione |
| B-131 | a compound of the formula I | procymidone |
| B-132 | a compound of the formula I | vinclozolin |
| B-133 | a compound of the formula I | acibenzolar-S-methyl |
| B-134 | a compound of the formula I | amisulbrom |
| B-135 | a compound of the formula I | anilazin |
| B-136 | a compound of the formula I | blasticidin-S |
| B-137 | a compound of the formula I | captan |
| B-138 | a compound of the formula I | captafol |
| B-139 | a compound of the formula I | chinomethionat |
| B-140 | a compound of the formula I | dazomet |
| B-141 | a compound of the formula I | debacarb |
| B-142 | a compound of the formula I | diclomezine |
| B-143 | a compound of the formula I | difenzoquat |
| B-144 | a compound of the formula I | difenzoquat methylsulfate |
| B-145 | a compound of the formula I | famoxadone |
| B-146 | a compound of the formula I | fenamidone |
| B-147 | a compound of the formula I | fenoxanil |
| B-148 | a compound of the formula I | fenpropidin |
| B-149 | a compound of the formula I | folpet |
| B-150 | a compound of the formula I | octhilinone |
| B-151 | a compound of the formula I | oxolinic acid |
| B-152 | a compound of the formula I | piperalin |
| B-153 | a compound of the formula I | probenazole |
| B-154 | a compound of the formula I | proquinazid |
| B-155 | a compound of the formula I | pyroquilon |
| B-156 | a compound of the formula I | quinoxyfen |
| B-157 | a compound of the formula I | triazoxid |
| B-158 | a compound of the formula I | tricyclazole |
| B-159 | a compound of the formula I | triforine |
| B-160 | a compound of the formula I | 5-chloro-7-(4-methylpiperidin-1-yl)- |
| | | 6-(2,4,6-trifluorophenyl)-[1,2,4]triazolo- |
| | | [1,5-a]pyrimidine |
| B-161 | a compound of the formula I | 2-butoxy-6-iodo-3-propylchromen-4-one |
| B-162 | a compound of the formula I | ferbam |
| B-163 | a compound of the formula I | mancozeb |
| B-164 | a compound of the formula I | maneb |
| B-165 | a compound of the formula I | metiram |
| B-166 | a compound of the formula I | metam |
| B-167 | a compound of the formula I | methasulphocarb |
| B-168 | a compound of the formula I | propineb |
| B-169 | a compound of the formula I | thiram |
| B-170 | a compound of the formula I | zineb |
| B-171 | a compound of the formula I | ziram |
| B-172 | a compound of the formula I | diethofencarb |
| B-173 | a compound of the formula I | flubenthiavalicarb |
| B-174 | a compound of the formula I | iprovalicarb |
| B-175 | a compound of the formula I | propamocarb |
| B-176 | a compound of the formula I | propamocarb hydrochloride |
| B-177 | a compound of the formula I | 3-(4-chlorophenyl)-3-(2-isopropoxy- |
| | | carbonylamino-3-methylbutyrylamino)- |
| | | propionic acid methyl ester |
| B-178 | a compound of the formula I | valiphenal |
| B-179 | a compound of the formula I | N-(1-(1-(4-cyanophenyl)ethanesulfonyl)- |
| | | but-2-yl)carbamic acid 4-fluorophenyl ester |
| B-180 | a compound of the formula I | dodine |
| B-181 | a compound of the formula I | dodine free base |
| B-182 | a compound of the formula I | iminoctadine |
| B-183 | a compound of the formula I | iminoctadine triacetate |
| B-184 | a compound of the formula I | iminoctadine tris(albesilate) |
| B-185 | a compound of the formula I | guazatine |
| B-186 | a compound of the formula I | guazatine acetate |
| B-187 | a compound of the formula I | kasugamycin |
| B-188 | a compound of the formula I | kasugamycin hydrochloride hydrate |
| B-189 | a compound of the formula I | polyoxine |
| B-190 | a compound of the formula I | streptomycin |
| B-191 | a compound of the formula I | validamycin A |
| B-192 | a compound of the formula I | binapacryl |
| B-193 | a compound of the formula I | dicloran |
| B-194 | a compound of the formula I | dinobuton |
| B-195 | a compound of the formula I | dinocap |
| B-196 | a compound of the formula I | nitrothal-isopropyl |
| B-197 | a compound of the formula I | tecnazen |
| B-198 | a compound of the formula I | fentin acetate |
| B-199 | a compound of the formula I | fentin chloride |
| B-200 | a compound of the formula I | fentin hydroxide |
| B-201 | a compound of the formula I | isoprothiolane |
| B-202 | a compound of the formula I | dithianon |
| B-203 | a compound of the formula I | edifenphos |
| B-204 | a compound of the formula I | fosetyl |
| B-205 | a compound of the formula I | fosetyl aluminum |
| B-206 | a compound of the formula I | iprobenfos |
| B-207 | a compound of the formula I | pyrazophos |
| B-208 | a compound of the formula I | tolclofos-methyl |
| B-209 | a compound of the formula I | chlorothalonil |
| B-210 | a compound of the formula I | dichlofluanid |
| B-211 | a compound of the formula I | dichlorophen |
| B-212 | a compound of the formula I | flusulfamide |
| B-213 | a compound of the formula I | hexachlorbenzene |
| B-214 | a compound of the formula I | pencycuron |
| B-215 | a compound of the formula I | pentachlorophenol and salts thereof |
| B-216 | a compound of the formula I | phthalide |
| B-217 | a compound of the formula I | quintozene |
| B-218 | a compound of the formula I | thiophanate methyl |
| B-219 | a compound of the formula I | tolylfluanid |
| B-220 | a compound of the formula I | N-(4-chloro-2-nitrophenyl)-N-ethyl- |
| | | 4-methylbenzenesulfonamide |
| B-221 | a compound of the formula I | phosphorous acid and its salts |
| B-222 | a compound of the formula I | sulfur |
| B-223 | a compound of the formula I | Bordeaux mixture |
| B-224 | a compound of the formula I | copper acetate |
| B-225 | a compound of the formula I | copper hydroxide |
| B-226 | a compound of the formula I | copper oxychloride |
| B-227 | a compound of the formula I | basic copper sulfate |
| B-228 | a compound of the formula I | biphenyl |
| B-229 | a compound of the formula I | bronopol |
| B-230 | a compound of the formula I | cyflufenamid |
| B-231 | a compound of the formula I | cymoxanil |
| B-232 | a compound of the formula I | diphenylamin |
| B-233 | a compound of the formula I | metrafenon |
| B-234 | a compound of the formula I | mildiomycin |
| B-235 | a compound of the formula I | oxine-copper |
| B-236 | a compound of the formula I | prohexadione-calcium |
| B-237 | a compound of the formula I | spiroxamin |
| B-238 | a compound of the formula I | tolylfluanid |
| B-239 | a compound of the formula I | N-(cyclopropylmethoxyimino-(6-difluoro- |
| | | methoxy-2,3-difluorophenyl)-methyl)- |
| | | 2-phenylacetamide |
| B-240 | a compound of the formula I | N′-(4-(4-chloro-3-trifluoromethylphenoxy)- |
| | | 2,5-dimethylphenyl)-N-ethyl-N-methyl- |
| | | formamidine |
| B-241 | a compound of the formula I | N′-(4-(4-fluoro-3-trifluoromethylphenoxy)- |
| | | 2,5-dimethylphenyl)-N-ethyl-N-methyl- |
| | | formamidine |
| B-242 | a compound of the formula I | N′-(2-methyl-5-trifluoromethyl-4-(3-tri- |
| | | methylsilanylpropoxy)phenyl)-N-ethyl- |
| | | N-methylformamidine |
| B-243 | a compound of the formula I | N′-(5-difluoromethyl-2-methyl-4-(3-tri- |
| | | methylsilanylpropoxy)phenyl)-N-ethyl- |
| | | N-methylformamidine |

The active compounds II specified above as component 2, their preparation, and their effect against fungal pathogens are widely known (cf.: http://www.hclrss.demon.co.uk/index.html); they are available commercially. The compounds with IUPAC nomenclature, their preparation, and their fungicidal activity are likewise known [cf. EP-A 226 917; EP-A 10 28 125; EP-A 10 35 122; EP-A 12 01 648; WO 98/46608; WO 99/24413; WO 03/14103; WO 03/053145; WO 03/066609; WO 04/049804].

### SYNTHESIS EXAMPLES

### 1) Synthesis of (4-fluorobenzoyl)formate

4-Fluorophenylmagnesium bromide (205 ml, 205 mmol, obtained by reaction of 4-fluorobromobenzene with magnesium turnings in tetrahydrofuran) was dropwise to a solution, cooled to −65° C., of diethyl oxalate (30.0 g, 205.2 mmol) in a mixture of tetrahydrofuran and diethyl ether (1:1, 300 ml). After the addition had ended, the mixture was stirred at this temperature for a further 30 min and then warmed to −25° C. After addition of aqueous hydrochloric acid (340 ml of a 1M solution), the organic phase was separated off and the aqueous phase was extracted with ethyl acetate (1×300 ml). The combined organic phases were washed with saturated sodium chloride solution (2×200 ml), dried over sodium sulfate, filtered and freed from the solvent. Purification by column chromatography (silica gel, hexane/dichloromethane 3:2) gave (4-fluorobenzoyl)formate as a yellow oil (35.0 g 87%).

H-NMR (300 MHz, CDCl₃): δ 8.10-8.05 (m, 2H), 7.20-7.16 (m, 2H), 4.44 (q, J=7.0 Hz, 2H) 1.42 (t, J=7.0 Hz, 3H).

### 2) Synthesis of [(2,5-dichlorothiophen-3-yl)methyl]triphenylphosphonium Chloride

Triphenylphosphane (2.1 g, 8.0 mmol) was added to a solution of 2,5-dichloro-3-(chloromethyl)thiophene (1.6 g, 8.0 mmol) in acetonitrile. The reaction solution was stirred at 82° C. for 24 hours and then cooled to room temperature, and the solvent was distilled off. The residue obtained was slurried with diethyl ether, and the product (2.4 g, 65%) was filtered off in the form of a colorless solid.

### 3) Synthesis of Ethyl 3-(2,5-dichlorothiophen-3-yl)-2-(4-fluorophenyl)acrylate

Potassium tert-butoxide (217 mg, 1.9 mmol) was added to a solution, cooled with ice, of [(2,5-dichlorothiophen-3-yl)methyl]triphenylphosphonium chloride (1.0 g, 2.1 mmol) in toluene (15 ml). After 30 min at this temperature, (4-fluorobenzoyl)formate (444 g, 2.2 mmol) in toluene was added dropwise, and after the addition had ended the mixture was warmed to room temperature over a period of one hour. Ethyl acetate (20 ml) was then added, and the reaction mixture was washed with saturated sodium chloride solution (2×10 ml). The organic phase was separated off, dried over sodium sulfate and filtered, and the solvent was removed under reduced pressure. The residue obtained was purified by column chromatography (silica gel, hexane/ethyl acetate 20:1). The appropriate fractions were combined, giving ethyl 3-(2,5-dichlorothiophen-3-yl)-2-(4-fluorophenyl)acrylate (290 mg, 37%) as a 2:1 mixture of trans to cis isomer in the form of a colorless oil.

H-NMR (300 MHz, CDCl₃): δ 7.76 (s, 1H), 7.19-7.09 (m, 4H), 5.79 (s, 1H) 4.26 (q, J=7.0 Hz, 2H), 1.29 (t, J=7.0 Hz, 3H).

### 4) Synthesis of (E)-3-(2,5-dichlorothiophen-3-yl)-2-(4-fluorophenyl)prop-2-en-1-ol

At −78° C. diisobutylaluminum hydride (4.4 ml of a 1M solution in toluene, 4.4 mmol) was added dropwise to a solution of ethyl 3-(2,5-dichlorothiophen-3-yl)-2-(4-fluorophenyl)acrylate (280 mg, 0.8 mmol) in anhydrous tetrahydrofuran (50 ml). The reaction solution was stirred between −78 and −60° C. for 25 min, and methanol was then added at −60° C. The resulting precipitate was filtered off and washed with methanol (10 ml), and the combined filtrates were freed from the solvent. Purification by column chromatography (silica gel, dichloromethane) and pooling of the appropriate fractions gave (E)-3-(2,5-dichlorothiophen-3-yl)-2-(4-fluorophenyl)prop-2-en-1-ol as a colorless oil (80 mg, 33%).

H-NMR (300 MHz, CDCl₃): δ 7.84 (s, 1H), 7.21-7.17 (m, 2H), 7.10-7.06 (m, 2H) 6.61 (s, 2H), 5.89 (s, 1H), 4.43 (d, J=8.0 Hz, 2H).

### 5) Synthesis of (E)-3-(2,5-dichlorothiophen-3-yl)-2-(4-fluorophenyl)acrylaldehyde

At 0° C., Dess-Martin periodinane (122 mg, 0.2 mmol) was added to a solution of (E)-3-(2,5-dichlorothiophen-3-yl)-2-(4-fluorophenyl)acrylaldehyde (80 mg, 0.2 mmol) in anhydrous dichloromethane (5 ml). Over a period of one hour, the reaction mixture was warmed to room temperature, diluted with ethyl acetate (10 ml) and washed with saturated sodium bicarbonate solution (2×10 ml). The organic phase was treated with saturated sodium chloride solution, dried over sodium sulfate, filtered and concentrated. The residue was purified by column chromatography (silica gel, hexane/ethyl acetate 10:1). The aldehyde was obtained when the appropriate fractions were combined and the solvent was removed (60 mg, 76%).

H-NMR (300 MHz, CDCl₃): δ 9.76 (s, 1H), 7.36 (s, 1H), 7.16 (s, 2H) 7.14 (s, 2H), 6.06 (s, 1H).

### 6) Synthesis of [(2SR,3RS)-3-(2,5-dichlorothiophen-3-yl)-2-(4-fluorophenyl)oxiran-2-yl)methanol

(E)-3-(2,5-dichlorothiophen-3-yl)-2-(4-fluorophenyl)acrylaldehyde (60 mg, 0.1 mmol) was dissolved in methanol (2 ml), and aqueous sodium hydroxide solution (0.01 mmol) and hydrogen peroxide (8 mg of a 50% strength aqueous solution, 0.2 mmol) were added at room temperature. After one hour at this temperature, the reaction mixture was cooled to 0° C., sodium borohydride (7 mg, 0.1 mmol) was added and, after thawing to room temperature, the mixture was stirred for a further hour. The reaction was then terminated by addition of saturated aqueous ammonium chloride solution (2 ml). After extraction with ethyl acetate (20 ml), the organic phase was washed with saturated sodium chloride solution (2×5 ml), dried over sodium sulfate, filtered and freed from the solvent. The residue obtained (56 mg) was used for the next reaction step without further purification.

H-NMR (300 MHz, CDCl₃): δ 7.53-7.48 (m, 2H), 7.13-7.07 (m, 2H), 6.82 (s, 1H), 4.05 (d, J=12.9 Hz, 1H), 3.89 (s, 1H), 3.68 (d, J=12.9 Hz, 1H).

### 7) Synthesis of [(2SR,3RS)-3-(2,5-dichlorothiophen-3-yl)-2-(4-fluorophenyl)oxiran-2-yl)methyl Methanesulfonate

Triethylamine (35 mg, 0.2 mmol) and methanesulfonyl chloride (30 mg, 0.2 mmol) was added to a solution, cooled with ice, of [(2SR,3RS)-3-(2,5-dichlorothiophen-3-yl)-2-(4-fluorophenyl)oxiran-2-yl)methanol (56 mg, 0.1 mmol) in dichloromethane (4 ml). The reaction mixture was warmed to room temperature and stirred for a further 18 hours, ethyl acetate was then added and the mixture was washed with saturated sodium bicarbonate solution (2×5 ml). The organic phase was dried over sodium sulfate and the solvent was removed under reduced pressure. The residue obtained was purified by column chromatography (silica gel, hexane/ethyl acetate 20:1). This gave, after pooling of appropriate fractions, [(2SR,3RS)-3-(2,5-dichlorothiophen-3-yl)-2-(4-fluorophenyl)oxiran-2-yl]methyl methanesulfonate (30 mg, 43%) in the form of a colorless oil.

H-NMR (300 MHz, CDCl₃): δ 7.50-7.45 (m, 2H), 7.15-7.09 (m, 2H), 6.85 (s, 1H), 4.68 (d, J=12.0 Hz, 1H), 4.21 (d, J=12.0 Hz, 1H), 3.94 (s, 1H), 2.80 (s, 3H).

### 8) Synthesis of 1-{[(2SR,3RS)-3-(2,5-dichlorothiophen-3-yl)-2-(4-fluorophenyl)oxiran-2-yl]methyl}-1H-1,2,4-triazole

A mixture of 1,2,4-triazole (15 mg, 0.2 mmol) and sodium hydride (5 mg, 0.2 mmol) in anhydrous N,N-dimethylformamide (1 ml) was stirred at room temperature for 15 min. A solution of [(2SR,3RS)-3-(2,5-dichlorothiophen-3-yl)-2-(4-fluorophenyl)oxiran-2-yl)methyl methanesulfonate (30 g, 0.05 mmol) in N,N-dimethylformamide (2 ml) was added, and the reaction solution was stirred at room temperature for five hours. The mixture was then diluted with ethyl acetate and washed with saturated sodium chloride solution (2×5 ml). The organic phase was dried over sodium sulfate and filtered, and the solvent was removed under reduced pressure. Purification by column chromatography (silica gel, hexane/ethyl acetate 1:1) gave the target compound (19 mg, 70%).

H-NMR (300 MHz, CDCl₃): δ 7.88 (s, 1H), 7.82 (s, 1H), 7.31-7.27 (m, 2H), 7.05-6.99 (m, 2H), 6.91 (s, 1H), 4.80 (d, J=15.5 Hz, 1H), 4.17 (d, J=15.5 Hz, 1H), 4.00 (s, 1H).

### Biological Section

### Greenhouse

### Preparation of Active Compound

The active compounds were prepared separately or jointly as a stock solution comprising 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the concentration of active compound stated below.

### Use Example 1

### Protective Activity Against Puccinia recondita on Wheat (Brown Rust of Wheat) (Puccrt P1)

Leaves of potted wheat seedlings were sprayed to runoff point with an aqueous suspension having the active compound concentration stated below. The next day, the treated plants were inoculated with a spore suspension of brown rust of wheat (Puccinia recondita). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and at 65 to 70% relative atmospheric humidity for a further 7 days. The extent of the rust fungus development on the leaves was then determined visually.

The plants treated with an aqueous active compound preparation comprising 250 ppm of the active compound 1-{[(2SR,3RS)-3-(2,5-dichlorothiophen-3-yl)-2-(4-fluorophenyl)oxiran-2-yl]methyl}-1H-1,2,4-triazole showed an infection of 0%, whereas the untreated plants were 90% infected.

### Use Example 2

### Activity Against Net Blotch of Barley Caused by Pyrenophora teres at 1 Day Protective Application (Pyrnte P1)

Leaves of potted barley seedlings were sprayed to runoff point with an aqueous suspension having the active compound concentration stated below. 24 hours after the spray coat had dried on, the test plants were inoculated with an aqueous spore suspension of Pyrenophora [syn. Drechslera] teres, the net blotch pathogen. The test plants were then placed in a greenhouse at temperatures between 20 and 24° C. and at 95 to 100% relative atmospheric humidity. After 6 days, the extent of the development of the disease was determined visually in % infection of the entire leaf area.

The plants treated with an aqueous active compound preparation comprising 250 ppm of the active compound 1-{[(2SR,3RS)-3-(2,5-dichlorothiophen-3-yl)-2-(4-fluorophenyl)oxiran-2-yl]methyl}-1H-1,2,4-triazole showed an infection of 15%, whereas the untreated plants were 90% infected.

1-11. (canceled)

12. A compound of formula I 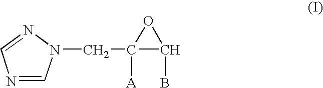
wherein,
A or B is a 5-membered heteroaryl selected from the group consisting of thienyl, furyl, pyrrolyl, pyrazolyl, imidazolyl, oxazolyl, thiazolyl, isoxazolyl and isothiazolyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
and the respective other substituent
A or B is phenyl which is optionally substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
or a plant-compatible acid addition salt or metal salt thereof.

13. The compound of claim 12, wherein said 5-membered heteroaryl is selected from the group consisting of thienyl, furyl, pyrrolyl, pyrazolyl, imidazolyl and thiazolyl.

14. The compound of claim 12, wherein said 5-membered heteroaryl is selected from the group consisting of thienyl and pyrazolyl.

15. The compound of claim 12, wherein said 5-membered heteroaryl is substituted by one to three of the following substituents: halogen, C₁-C₄-alkyl, C₁-C₄-alkoxy, C₁-C₄-haloalkyl or C₁-C₄-haloalkoxy.

16. The compound of claim 15, wherein said 5-membered heteroaryl is substituted by one to three of the following substituents: halogen, C₁-C₄-alkyl or C₁-C₄-alkoxy.

17. The compound of claim 12, wherein said respective other substituent A or B, which is different from said 5-membered heteroaryl, is phenyl which is substituted by one to three of the following substituents: halogen, C₁-C₄-alkyl, C₁-C₄-alkoxy, C₁-C₄-haloalkyl or C₁-C₄-haloalkoxy.

18. The compound of claim 17, wherein said phenyl is substituted by halogen, C₁-C₄-alkyl or C₁-C₄-alkoxy.

19. A composition comprising a solid or liquid carrier and a compound of the formula I of claim 12 and/or an acid addition salt or metal salt thereof.

20. Seed comprising at least one compound of the formula I of claim 12 and/or an acid addition salt or metal salt thereof.

21. A method for controlling phytopathogenic fungi wherein the fungi or the materials, plants, the soil or seed to be protected against fungal attack are/is treated with an effective amount of a compound of the formula I 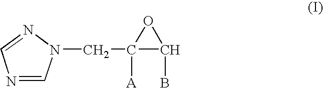
wherein,
A or B is a 5-membered heteroaryl selected from the group consisting of thienyl, furyl, pyrrolyl, pyrazolyl, imidazolyl, oxazolyl, thiazolyl, isoxazolyl and isothiazolyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
and the respective other substituent
A or B is phenyl which is optionally substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
or a plant-compatible acid addition salt or metal salt thereof.

22. The method of claim 21, wherein said 5-membered heteroaryl is selected from the group consisting of thienyl, furyl, pyrrolyl, pyrazolyl, imidazolyl and thiazolyl.

23. The method of claim 21, wherein said 5-membered heteroaryl is selected from the group consisting of thienyl and pyrazolyl.

24. The method of claim 21, wherein said 5-membered heteroaryl is substituted by one to three of the following substituents: halogen, C₁-C₄-alkyl, C₁-C₄-alkoxy, C₁-C₄-haloalkyl or C₁-C₄-haloalkoxy.

25. The method of claim 24, wherein said 5-membered heteroaryl is substituted by one to three of the following substituents: halogen, C₁-C₄-alkyl or C₁-C₄-alkoxy.

26. The method of claim 21, wherein said respective other substituent A or B, which is different from said 5-membered heteroaryl, is phenyl which is substituted by one to three of the following substituents: halogen, C₁-C₄-alkyl, C₁-C₄-alkoxy, C₁-C₄-haloalkyl or C₁-C₄-haloalkoxy.

27. The method of claim 26, wherein said phenyl is substituted by halogen, C₁-C₄-alkyl or C₁-C₄-alkoxy.
